(19) United States
(12) Patent Application Publication
(10) Pub. No.: US 2002/0071872 A1
(43) Pub. Date: Jun. 13, 2002

(54) Laxative composition

(76) Inventors: Gerard P. McNally, Berwyn, PA (US); Charles E. Pendley II, Abington, PA (US); Brid T. Burruano, King of Prussia, PA (US); Nancy B. Rainey, Villanova, PA (US); Steven A. Jacobs, Hatfield, PA (US); Shun Por Li, Lansdale, PA (US); Michael E. Mertzman, New Jersey, PA (US); David W. Wynn, Abington, PA (US); Anthony J. Le Storti, Doylestown, PA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 09/924,319

(22) Filed: Aug. 8, 2001

Related U.S. Application Data
(63) Continuation-in-part of application No. 09/390,813, filed on Sep. 7, 1999, now abandoned.

(51) Int. Cl. A61K 9/16; A61K 9/50; A61K 31/16

(52) U.S. Cl. 424/490; 514/627

(57) ABSTRACT

The present invention provides a composition comprising a) a laxative selected from the group consisting of bisacodyl and enteric coated vanilloid compounds; and b) simethicone in an amount effective to enhance the efficacy of the laxative. The simethicone enhances the transit of the laxatives through the small bowel.

### CROSS REFERENCE TO RELATED APPLICATION

This application is a continuation-in-part of U.S. patent application Ser. No. 09/390,813, filed Sep. 7, 1999, which is hereby incorporated by reference.

### FIELD OF THE INVENTION

The present invention relates to a laxative composition, more particularly a laxative composition containing a therapeutic amount of simethicone or dimethicone.

### BACKGROUND OF THE INVENTION

Laxative compositions are typically categorized by the mechanism in which they work, such as bulk, saline, stool softener, lubricant, or stimulant as per the U.S. Food and Drug Administration's monograph, Laxatives, Martindale, page 1070; or Goodman and Gilman page 914. Bulk laxatives contain materials such as psyllium, cellulose, polycarbophil, bran, karaya and malt soup extract. Saline laxatives, such as magnesium, hydroxide, sulfate, phosphate, and citrate salts act by drawing water into the intestines. Stool softeners include docusate salts and mineral oils. Lubricant laxatives include mineral oil, and certain digestible plant oils. Lubricants coat the fecal contents, preventing excess absorption of water in the colon. Stimulants include bisacodyl, cascara sagrada, senna, aloe, castor oil and dehydrocholic acid. Stimulant laxatives work to increase intestinal motility by either increasing peristaltic activity as a result of local irritation, or by selective stimulation of the nerves, which activate intestinal smooth muscle.

While the above materials are effective laxative materials, there is a continuing need to enhance the performance of these materials by providing faster onset of action and superior efficacy.

In addition to the above compounds, U.S. Pat. No. 5,418,220 discloses simethicone as a treatment for constipation. In the patent example, one teaspoon of a dimethicone suspension (approximately 33% in glycerin stearate and water) resulted in laxation in a 2 year old patient approximately two hours after administration.

### SUMMARY OF THE INVENTION

It has been surprisingly found that the incorporation of simethicone enhances the efficacy of laxatives, in particular bisacodyl and enteric coated vanilloid compounds, such as capsaicin. Accordingly, the invention provides a composition comprising: a) a laxative selected from the group consisting of bisacodyl and enteric coated vanilliod compounds; and b) simethicone in an amount effective to enhance the efficacy of the laxative.

In a second embodiment, the invention provides a method of treating constipation, diabetic gastro-paresis, or gastro-esophageal reflux disorder, or of improving gastro-intestinal motility, comprising administering to a human an effective amount of a composition comprising: a) a laxative selected from the group consisting of bisacodyl and enteric coated vanilloid compounds; and b) simethicone in an amount effective to enhance the efficacy of the laxative.

In a third embodiment, the invention provides a method for enhancing the efficacy of a laxative selected from the group consisting of bisacodyl and enteric coated vanilloid compounds comprising providing therewith an effective amount of simethicone.

### DETAILED DESCRIPTION OF THE INVENTION

Dimethicone is a well known pharmaceutical material consisting of linear siloxane polymers containing repeating units of the formula {—(CH₂)₂SiO}ₙ stabilized with trimethylsiloxy end blocking units of the formula [(CH₃)₃SiO—]. Simethicone is the mixture of dimethicone and silicon dioxide. For the purposes of this invention, the two materials may be used interchangeably.

The level of simethicone or dimethicone in the present composition is effective to enhance the effect of the laxative, i.e., bisacodyl or enteric coated vanilloid. Generally this level is from about 10 mg to about 500 mg, preferably from about 25 to about 300 mg, and most preferably from about 50 mg to about 125 mg per dosage unit. Higher levels of simethicone can also be employed such as levels as high as 2250 mg per oral dosage unit. If used rectally, high concentrations, as high as needed for a good enema, can be envisaged as based on use in topicals (e.g. 33% of the volume to be administered per dose unit.)

The laxative is selected from bisacodyl, enteric coated, biologically active vanilloid compounds, and mixtures thereof. The level of laxative is the amount necessary to provide the desired effect, which is generally from about 1.0 mg to about 100 mg, preferably from about 1.0 mg to about 50 mg, and most preferably from about 1.0 mg to about 15 mg per dosage unit for bisacodyl, and alternately from about 5 mg to about 25 mg for vanilloid compounds.

The vanilloid compound may be a natural or synthetic compound, including pharmaceutically acceptable salts, analogues and/or derivatives thereof. Also included are both crude extracts and purified extracts of active vanilloid compounds. Examples of natural vanilloid compounds suitable for use in the present invention include both the crude extracts and the purified extracts of: capsicum, cayenne pepper, black pepper, paprika, cinnamon, clove, mace, mustard, ginger, tumeric, papaya seed and the cactus-like plant Euphorbia resinifera. Synthetic vanilloid compounds such as synthetic capsaicin as defined in WO96/40079 are also suitable.

In one embodiment the active vanilloid compound is selected from capsaicin ((E)-(N)-[(4-hydroxy-3-methoxyphenyl)-methyl]-8-methyl-6-nonenamide); eugenol (2-methoxy-4-(2-propenyl)phenol); zingerone (4-(4-hydroxy-3-methoxyphenyl)-2-butanone); curcumin (1,7-bis(4-hydroxy-3-methoxyphenyl)-1,6-heptadiene-3,5-dione); piperine (1-[5-(1,3-benzodioxol-5-yl)-1-oxo-2,4-pentadienyl]piperidine); resiniferatoxin (6,7-deepoxy-6,7-didehydro-5-deoxy-21-dephenyl-21-(phenylmethyl)-20-(4-hydroxy-3-methoxybenzeneacetate)), pharmaceutically effective salts, analogues, derivatives, and equivalents thereof

Most preferably, the enteric coated vanilloid compound is enteric coated capsaicin.

A dosage unit can be one tablet, capsule, or suppository, one teaspoonful of a liquid, or one single portion of any other suitable delivery form. The present invention can be delivered as a tablet, a chewable tablet, a liquid drink, a suppository or other pharmaceutically acceptable forms. Oral delivery forms are preferred.

Commonly known pharmaceutically acceptable additives for orally-administered drugs such as enteric polymers, taste-masking polymers, binders, sweeteners, flavoring agents, dispersants, buffering agents and the like may be included in amounts that do not adversely affect the novel properties of the formulation described and claimed herein.

In one embodiment the enteric coated vanilloid compound is in the form of a coated particle. The core of the particle may comprise pure, crystalline vanilloid compound, or a mixture of active ingredient with optional ingredients, such as binders, excipients and the like known in the art. The core may be formed using a variety of well known granulation methods, including high sheer wet granulation, spray drying, and fluid bed granulation (including rotary fluid bed granulation). Preferably, the particle core is made by fluid bed granulation. Suitable binders include microcrystalline cellulose, calcium phosphates, dextrates. Suitable dispersants include croscarmellose sodium, methylcellulose, hydroxymethylcellulose, hydroxypropylmethylcellulose, hydroxyethylcellulose and the like. Suitable sweeteners include sugar, sorbitol, saccharin, mannitol, glucose, aspartame, sucralose and the like. Flavoring agents include peppermint, spearmint, cinnamon, vanilla and the like. A more complete listing of appropriate additives can be found in numerous publications including Remington's Encyclopedia.

A polymeric coating comprising an enteric polymer covers the core. The enteric polymer may be selected from any one of a variety of known enteric polymers, such as hydroxypropyl methylcellulose phthalate, hydroxypropyl methylcellulose acetate succinate, cellulose acetate phthalate , polyvinylacetate phthalate, and polymethacrylate-based polymers such as poly(methacrylic acid, methyl methacrylate) 1:2 (commercially available from Rohm Pharma GmbH as Eudragit S polymers), and poly(methacrylic acid, methyl methacrylate) 1:1 (commercially available from Rohm Pharma GmbH as Eudragit L polymers). Combinations of enteric polymers may also be used.

Particle coating may be carried out by known procedures such as described in for example U.S. Pat. Nos. 3,196,827, 3,253,944, 5,814,332, 5,409711, 5,489436, 4,851,226, 5,773031.

The present invention is surprising and unexpected in that PCT EP95/00973 previously disclosed that simethicone is effective in association or affinity to the surface structure of the GI tract. The PCT patent application teaches that due to the increased adhesion properties of dimethicone the residence time of an active ingredient in a region of the GI tract can be substantially prolonged if dimethicone is used as a transport or carrier system.

As used herein diabetic gastro-paresis is defined as the dilation of the stomach with gastric retention seen in diabetics, commonly seen in association with severe acidosis or coma, Stedmans Medical Dictionary. Gastro-esophogeal reflux disorder (GERD) is defined as the regurgitation of the contents of the stomach into the esophagus, possibly into the pharynx where they can be aspirated between the vocal cords and down into the trachea; providing symptoms of burning pain and acid taste result; pulmonary complications of aspirations are dependent upon the amount content and acidity of the aspirate. Id.

The following examples are provided to further illustrate the claimed invention, but not limit the invention to the examples provided below.

### EXAMPLE 1

A study was performed to characterize and compare the effects of bisacodyl, simethicone and combinations of bisacodyl and simethicone on small bowel transit time. Small intestinal transit time was studied as a surrogate for Taxation in the rat. Leng-Peschlow, E., “Effect of Sennosides A+B and Bisacodyl on rat large intestine”, Pharmacology, vol. 38 (1989), 310-318 (1989). Observed increased fecal output over the range of 10-100 mg/kg of bisacodyl given orally, large intestinal transit was significantly stimulated by 50 mg/kg bisacodyl given orally. The prokinetic efficacy of bisacodyl in the small intestine may differ from that observed in the large intestine by Leng-Psechlow. For the small intestinal transit model a sub-therapeutic dose of 25mg/kg bisacodyl was chosen.

Rats were dosed with a suspension of powdered charcoal, which served as a non-absorbable marker. The rats were also dosed with bisacodyl USP 23 (ZetaPharm, Inc.) and simethicone supplied as MYLICON® drops and activated charcoal (Sigma Chemical). Six treatments were compared a control; simethicone 15 mg/kg; bisacodyl 25 mg/kg; and bisacodyl 25 mg/kg and simethicone at 5, 10 or 15 mg/kilogram.

Dosing preparations of charcoal suspension (10 weight percent) were made freshly each day by adding dry powder to 0.5 percent methylcellulose in water and stirring. Dosing preparations were made each day by adding the appropriate quantity of bisacodyl and/or simethicone drops to the charcoal suspension. All treatments were administered orally, using the dose volume of 10 milliliters per kilogram.

Sixty minutes after dosing, the rats were sacrificed and small bowel transit rate was determined by identifying the charcoal marker and measuring its distance from the pylorus.

Results reveal that small bowel motility was greater in rats treated with bisacodyl and simethicone combinations than in rats treated with either bisacodyl or simethicone alone. The results are presented below.

| | Mean % | Std. Error of | |
|---|---|---|---|
| Treatment | traveled | Mean | % Increase |
| Vehicle Control | 79.9 | 2.1 | — |
| Simethicone 15 mg/kg | 79.1 | 2.0 | −1 |
| Bisacodyl 25 mg/kg | 80.7 | 2.0 | 1 |
| Bisacodyl 25 mg/kg + | 90.3 | 2.5 | 13 |
| Simethicone 5 mg/kg | | | |
| Bisacodyl 25 mg/kg + | 97.0 | 1.7 | 21 |
| Simethicone 10 mg/kg | | | |
| Bisacodyl 25 mg/kg + | 94.4 | 2.0 | 18 |
| Simethicone 15 mg/kg | | | |

The results reveal that although small bowel transit was not different from control in rats treated with simethicone alone or bisacodyl alone, small bowel transit definitely increased in rats treated with the combination. The observed increases in small bowel transit were sizable (13 to 21 percent increase compared to the control) and the result was also statistically significant (p less than 0.05).

### EXAMPLE 2

Preparation of Chewable Tablets Containing Bisacodyl and Simethicone

PART A.

1) 700 gm of granular tricalcium phosphate (Tritab®, Rhone-Poulenc, Shelton, Conn.) is added to the mixing bowl of a Kitchen Aid mixer.

2) While mixing at low speed, over a period of 5 minutes add 200 gm of simethicone, USP.

3) Continue mixing at low speed for an additional 5 minutes.

4) Add 2.5 gm of silicon dioxide and mix an additional 5 minutes.

PART B.

Preparation of enteric coated bisacodyl particles.

1. Rotogranulation.

Combine 0.52 kg. of Bisacodyl, 0.24 kg. of Hydroxypropyl Methylcellulose (grade Methocel E5) and 3.24 kg. of Lactose impalpable in a rotor granulator bowl. Rotor granulate by spraying water (approx. 1.0 kg.) at a rotor speed of 500 RPM. Dry the rotogranulated particles to a product temperature of 30°-35° C. after decreasing rotor speed to 250 RPM.

2. Particle Coating.

Coat the particles produced in Step A in a Wurster Coating apparatus. The polymer coating solution should consist of the following; approximately 35% by weight aq. dispersion of Eudragit L30D containing approx. 2.5% Triethyl citrate. Apply 10-40% by weight polymer to the particles. Maintain product temperature at about 30-32° C. during the coating step. A further tastemasking coat is then applied as follows. The polymer coating solution should consist of 10% by weight solution of cellulose acetate 398-10, (39.8%acetyl content; 10 seconds viscosity) and hydroxypropylcellulose (Klucel EF) where the ratio of CA to HPC is 70/30. The solvent used is an 80/20 mixture of acetone/methanol. Apply a 5-10% by weight polymer coat to the particles. Maintain a product temperature at about 40-42° C. during coating.

PART C.

1) Blend 89 gm of the free flowing granular intermediate from Part A. with 7.34 g of coated bisacodyl, 98 gm of Dextrates, 7.5 gm granular sorbitol, 0.6 gm peppermint flavor, and 0.5 gm stearic acid.

2) Compress the blend using 5/8″ FFBE tooling to a tablet weight of 1287 mg

### EXAMPLE 3

Preparation of Swallowable Film Coated Tablets Containing Bisacodyl and Simethicone.

| | Qty |
|---|---|
| Ingredient | (mg/tab) |
| PART I-concentrate | |
| Dibasic calcium phosphate, NF, Anhydrous, granular | 500 |
| (DiTab ® ) | |
| Simethicone, USP | 125 |
| Tribasic calcium phosphate, NF, Anhydrous, Powder | 25 |
| Dibasic calcium phosphate, NF, Anhydrous, granular Powder | 90 |
| (Fujicalin ® SG) | |
| PART II-Bisacodyl/Excipient/Binder system | |
| Bisacodyl, USP | 5 |
| Microcrystalline cellulose, NF (MCC) | 205 |
| Croscarmellose sodium, NF | 30 |
| PART III-Lubricant | |
| Magnesium Stearate, NF | 4 |
| PART VI-Enteric Coating Step | |
| Eudragit ® S100 | 140 |

PART 1.

A concentrate comprised of granular anhydrous dibasic calcium phosphates, and simethicone is prepared by adding simethicone compound, USP to a moving bed of granular dibasic calcium phosphate so that the simethicone is distributed evenly and the granular calcium phosphate particle size remains essentially unchanged. The bed is kept in motion by low shear mixers such as fluid bed, Nauta, PK without intensifier bar, pin mixer, or ribbon mixer. The granulation may then be screened through a No. 20 US Std screen (˜840 micron).

PART 2.

Bisacodyl is added with low shear blending until the active ingredient is uniformly dispersed in the Simethicone blend. Excipients including a disintegrant are then added with low shear blending which imparts uniform distribution of the active within a binding matrix of limited compositional range.

PART 3.

The final addition step is to add a lubricant.

The blend is compressed into tablets using a rotary tablet press.

PART 4.

Tablets are then enteric film coated in a coating pan with an Eudragit S100 dispersion to a weight increase of approximately 5-25%.

We claim:

1. A composition comprising:
a) a laxative selected from the group consisting of bisacodyl and enteric coated vanilloid compounds; and
b) simethicone in an amount effective to enhance the efficacy of the laxative.

2. The composition of claim 1, wherein the vanilloid compound is capsaicin.

3. The composition of claim 1 formulated for oral administration.

4. The composition of claim 1 comprising about 10 mg to about 500 mg per dose of simethicone.

5. The composition of claim 1 wherein the laxative is bisacodyl present in an amount of from about 1 mg to about 15 mg per dose.

6. A method of treating constipation comprising administering to a human an effective amount of a composition comprising:
a) a laxative selected from the group consisting of bisacodyl and enteric coated vanilloid compounds; and
b) simethicone in an amount effective to enhance the efficacy of the laxative.

7. The method of claim 6, wherein the vanilloid compound is capsaicin.

8. A method of improving gastrointestinal motility in a human comprising administering an effective amount of a composition comprising:
a) a laxative selected from the group consisting of bisacodyl and enteric coated vanilloid compounds; and
b) simethicone in an amount effective to enhance the efficacy of the laxative.

9. The method of claim 8, wherein the vanilloid compound is capsaicin.

10. A method of treating diabetic gastro-paresis comprising administering to a human an effective amount of a composition comprising:
a) a laxative selected from the group consisting of bisacodyl and enteric coated vanilloid compounds; and
b) simethicone in an amount effective to enhance the efficacy of the laxative.

11. The method of claim 10, wherein the vanilloid compound is capsaicin.

12. A method of treating gastro-esophageal reflux disorder comprising administering to a human an effective amount of a composition comprising:
a) a laxative selected from the group consisting of bisacodyl and enteric coated vanilloid compounds; and
b) simethicone in an amount effective to enhance the efficacy of the laxative.

13. The method of claim 12, wherein the vanilloid compound is capsaicin.

14. A method for enhancing the efficacy of a laxative selected from the group consisting of bisacodyl and enteric coated vanilloid compounds comprising providing therewith an effective amount of simethicone.

15. The method of claim 14 wherein the laxative and simethicone are administered orally.

16. The method of claim 14 wherein the amount of simethicone provided is from about 10 mg to about 500 mg per dosage unit.

17. The method of claim 14, wherein the vanilloid compound is capsaicin.
